(19) United States
(12) Patent Application Publication
(10) Pub. No.: US 2007/0055339 A1
(43) Pub. Date: Mar. 8, 2007

(54) Staged stent delivery systems

(76) Inventors: William R. George, Santa Cruz, CA (US); Frank P. Becking, Palo Alto, CA (US)

Correspondence Address: CARDIOMIND, INC., 257 HUMBOLDT COURT, SUNNYVALE, CA, 94089, US

(21) Appl. No.: 11/211,129

(22) Filed: Aug. 23, 2005

(51) Int. Cl. A61F 2/06

(52) U.S. Cl. 623/1.11

(57) ABSTRACT

Medical devices and methods for delivery or implantation of prostheses within hollow body organs and vessels or other luminal anatomy are disclosed. The subject technologies may be used in the treatment of atherosclerosis in stenting procedures or a variety of other procedures. The various systems described employ self expanding stent restrained by tubular restraints. The systems are configured to reduce restraint actuation force relative to simple-sheath based stent delivery systems by actuation in a staged fashion.

### BACKGROUND OF THE INVENTION

Implants such as stents and occlusive coils have been used in patients for a wide variety of reasons. One of the most common “stenting” procedures is carried out in connection with the treatment of atherosclerosis, a disease which results in a narrowing and stenosis of body lumens, such as the coronary arteries. At the site of the narrowing (i.e., the site of a lesion) a balloon is typically dilatated in an angioplasty procedure to open the vessel. A stent is set in apposition to the interior surface of the lumen in order to help maintain an open passageway. This result may be effected by means of a scaffolding support alone or by virtue of the presence of one or more drugs carried by the stent to aide in the prevention of restenosis.

Various stent designs have been developed and used clinically, but self-expandable and balloon-expandable stent systems and their related deployment techniques are now predominant. Examples of self-expandable stents currently in use are the Magic WALLSTENT® stents and Radius stents (Boston Scientific). A commonly used balloon-expandable stent is the Cypher® stent (Cordis Corporation). Additional self-expanding stent background is presented in: “An Overview of Superelastic Stent Design,” Min. Invas Ther & Allied Technol 2002: 9(3/4) 235-246, “A Survey of Stent Designs,” Min. Invas Ther & Allied Technol 2002: 11(4) 137-147, and “Coronary Artery Stents: Design and Biologic Considerations,” Cardiology Special Edition, 2003: 9(2) 9-14, “Clinical and Angiographic Efficacy of a Self-Expanding Stent” Am Heart J 2003: 145(5) 868-874.

Because self-expanding prosthetic devices need not be set over a balloon (as with balloon-expandable designs), self-expanding stent delivery systems can be designed to a relatively smaller outer diameter than their balloon-expandable counterparts. As such, self-expanding stents may be better suited to reach the smallest vasculature or to achieve access in more difficult cases.

Sheath-based stent delivery systems are not only generally cost effective, but they also typically offer a highly space efficient delivery device solution for self-expanding stents. Yet, in situations where elastic or superelastic material stents are employed at high expansion ratios (i.e., 5 to 10×), the stents generate substantial in-sheath forces. It is important to minimize delivery system internal friction in order that the tubular member restraining the stent can be withdrawn from the same without the need for increasingly large input forces that can damage system components. What is more, high withdrawal forces are undesirable under circumstances of non-mechanically assisted user actuation.

Even when a sheath-based stent delivery system is suitable for use given a particular stent, problems can be encountered that involve the ability to accurately deliver the prosthesis as desired. In efforts to provide a system that is able to more accurately place a stent at a desired location, U.S. Pat. No. 5,201,757 describes a system in which a two-part sheath moves proximally and distally off of a stent to effect release. The system is described as one in which the stent is first allowed to expand along a medial region. Then, stent placement is adjusted. Finally, the tubular restraint is moved to ultimately deploy the stent.

Manipulation of the '757 design requires advancing the distal end of the delivery guide to move its distal sheath a significant length off of the stent. Forward motion on such a scale during stent deployment is not advisable from the perspective of vessel damage and may not even be possible in distal vasculature. Another issue with the operation of the device in the '757 patent is that no mechanism seems to exist to ensure that both halves of the tubular restraint are pushed/pulled free of the stent in equal fashion in order to expose the middle of the stent. Device operation seems to assume substantially equal static and dynamic friction along each side of the stent to effect such action. Should the system fail to operate as desired and the proximal end of the stent is deployed first, a serious problem exists. Under such circumstances, emergency withdrawal would not appear to be an option, given that the proximal to distal deployment will have exposed the wrong end of the stent for such a procedure. Instead, distal to proximal stent deployment (where the distal end of the stent opens first) is often preferred from a safety perspective since the distal or forward-facing crowns of the stent can be collapsed and withdrawn into whatever guiding or delivery catheter may be employed.

In view of the above considerations, there exists a continued interest in offering improved delivery systems that employ a tubular stent restraining member. The systems of the present invention address at least some of the above-referenced problems with known devices. In addition, those with skill in the art may appreciate further benefits or advantages of the subject invention.

### SUMMARY OF THE INVENTION

One aspect of the present invention employs a two-part tubular stent restraint. Unlike known devices using a two-part or split tube configuration for stent hold-down and release, the purpose of the split design in the preset invention is to moderate frictional forces between the tubular restraining member(s) and the stent. By only having to break-away static friction and continue withdrawal of a tubular member restraining a portion of the stent, lower actuation forces can be achieved relative to a sheath that fully covers a stent. Alternatively, the advantages offered in reduced frictional forces may be employed not to decrease actuation force, but instead to deliver larger/longer stents with radial force characteristics the would otherwise prohibit delivery using a tubular restraining member.

Systems according to the present invention may be unidirectionally actuated. That is to say, the systems may be actuated by simply withdrawing one or more members, rather than advancing a portion of the device to release the stent. Still, in certain variations of the invention, final release of a stent may be completed by slight or inconsequential forward (distally-directed) component motion.

Some variations of the invention effect multi-stage stent deployment with a single user input action. Stated otherwise, the user need only pull back a sheath, tubular restraint pull wire, etc., (at a handle or otherwise) and staged or sequential release activity will occur at a distal, stent-carrying portion of the device to release the stent.

One variation of the invention employs a two-piece tubular restraint. A proximal portion of the restraint is configured such that is it is able to slide a stent back over an inner member and out of a distal portion of the restraint. Then, after the stent has encountered a blocker or stop feature, continued actuation draws the proximal restraint off of the stent. In this manner, single-action actuation effects two-stage stent deployment.

Another set of systems according to the present invention likewise relies on sliding the stent backward (proximally) with a restraint and continued proximal movement of the restraint to delivery a stent. Yet, within this group of delivery systems, proximal movement of the restraint and stent releases or unlatches a radially expandable restraint member. Once freed, expansion of sections or segments of the restraint may, in turn, aid in pushing off the rest of the restraint or simply reduce the aggregate force the stent exerts on the restraint (thus reducing frictional forces between the members).

In order to facilitate restraint withdrawal, the proximal end of the stent is captured in one manner or another. In one approach, a proximal end of the stent is radially restrained by a relatively short proximal tubular restraint (a “mini-sheath”) underlying the releasable restraint. After the outer releasable restraint is drawn to a point in which it will not interfere with final stent deployment, the inner tubular restraint is then withdrawn with a stop or blocker axially stabilizing the stent. Alternatively, the blocker or stop section may be advanced out of the mini-sheath so long as the amount of motion required would not compromise patient safety.

In another instance, the stent may be captured at a proximal end by a partial restraining band, or the like fixed to the core member and/or stop member. In which case, final deployment of the stent may occur by withdrawing the core member together with the band (relying on the interaction between the stent and vessel wall to anchor the stent). Alternatively, the band may be sized to allow the stent to slip out of its final confinement (without deleteriously affecting desired stent placement).

In yet another approach to multi-stage deployment, two or more distally directed tubular restraint members are employed. The outermost of them extends farthest (generally to the end) of the stent. The innermost of the restraint members extends the shortest distance over the length of the stent. By withdrawing the outer member first, only the portion of the stent that it radially restraints is released. Then, subsequent restraint member(s) are pulled to complete stent release and deployment.

Common to each of the systems is that they offer relative reduction of tubular restraint retraction force by breaking-up the required load. Importantly, this improvement is accomplished while offering stent release in a distal-to-proximal fashion. Still further, problematic advancement of delivery system components is avoided in effecting stent delivery. Hence, the present invention offers improvement in any of a number of areas. Realizing such improvements may be especially useful in the context of small-vessel or other body lumen applications. However, the improvement(s) may be useful in a variety of settings. In addition, it is noted that those with skill in the art may appreciate further advantages or benefits of the present invention.

### DEFINITIONS

The term “stent” as used herein refers to any coronary artery stent, other vascular prosthesis, or other radially expanding or expandable prosthesis or scaffold-type implant suitable for the noted treatments or otherwise. Exemplary structures include wire mesh or lattice patterns and coils, though others may be employed in the present invention.

A “self-expanding” stent as used herein is a scaffold-type structure (serving any of a number of purposes) that expands from a reduced-diameter (be it circular or otherwise) configuration to an increased-diameter configuration by elastic or pseudoelastic recovery in response to removal of a restraining member. Accordingly, when held by the restraint, the stent strains or presses against the inner wall of the restraint structure. As such, neither the alloy nor the delivery system is configured so that the stent will retain its shape within the body without restraint. In-other words, where an alloy such as Nitinol is used in a stent according to the present invention, its Af temperature is at body temperature or below (i.e., less than or equal to about 37 degrees C.)

A “wire” as used herein generally comprises a common metallic member. However, the wire may be coated or covered by a polymeric material (e.g., with a lubricious material such as TEFLON®, i.e., PolyTetraFluoroEthelyne—PTFE) or otherwise. Still further, the “wire” may be a hybrid structure with metal and a polymeric material (e.g., Vectran™, Spectra™, Nylon, etc.) or composite material (e.g., carbon fiber in a polymer matrix). The wire may be a filament, bundle of filaments, cable, ribbon or in some other form. It is generally not hollow.

A “core” wire as referred to herein is a member internal to an outer member, such as a tubular member. As a core wire, the member fills or at least substantially fills all of the interior space of the tubular member.

An “inner member” as disclosed herein may be a core member or a core wire or be otherwise configured.

A “hypotube” or “hypotubing” as referred to herein means small diameter tubing in the size range discussed below, generally with a thin wall. The hypotube may specifically be hypodermic needle tubing. Alternatively, it maybe wound or braided cable tubing, such as provided by Asahi Intec Co., Ltd. Or otherwise. As with the “wire” discussed above, the material defining the hypotube may be metallic, polymeric or a hybrid of metallic and polymeric or composite material.

A “sleeve” as referred to herein may be made of such hypotubing or otherwise. The sleeve may be a tubular member, or it may have longitudinal opening(s). It is an outer member, able to slidingly receive and hold at least a portion of an inner member.

An “atraumatic tip” may comprise a plurality of spring coils attached to a tapered wire section. At a distal end of the coils typically terminate with a bulb or ball that is often made of solder. In such a construction, the coils and/or solder are often platinum alloy or another radiopaque material. The coils may also be platinum, or be of another material. In the present invention, the wire section to which the coils are attached may be tapered, but need not be tapered. In addition, alternate structures are possible. In one example, the atraumatic tip may comprise a molded tantalum-loaded 35 durometer Pebax™ tip. However constructed, the atraumatic tip may be straight or curved, the latter configuration possibly assisting in directing or steering the delivery guide to a desired intravascular location.

To “connect” or to have or make a “connection” between parts refers to fusing, bonding, welding (by resistance, laser, chemically, ultrasonically, etc.), gluing, pinning, crimping, clamping or otherwise mechanically or physically joining, attaching or holding components together (permanently or temporarily).

### BRIEF DESCRIPTIONS OF THE DRAWINGS

The figures shown herein are not necessarily drawn to scale, with some components and features being exaggerated for clarity. Each of the figures diagrammatically illustrates aspects of the invention. Of these:

FIG. 1 shows a heart in which its vessels may be the subject of one or more angioplasty and stenting procedures;

FIG. 2A shows an expanded stent cut pattern as may be used in producing a stent according to a first aspect of the invention; FIG. 2B shows a stent cut pattern for a second stent produced according to another aspect of the present invention;

FIG. 3A shows an expanded stent cut pattern as may be used in producing a stent according to a first aspect of the invention; FIG. 3B shows a stent cut pattern for a second stent produced according to another aspect of the present invention;

FIGS. 4A-4L illustrate stent deployment methodology to be carried out with the subject delivery guide member;

FIG. 5 provides an overview of a delivery system incorporating a tubular member according to the present invention;

FIGS. 6A-6C show an exemplary variation of a subject delivery system employing a first sequential stent release approach;

FIGS. 7A-7D show another exemplary variation of a subject delivery system employing an expandable restraint approach;

FIG. 8 illustrates a delivery device system like that in FIGS. 7A-7D without a proximal stent stabilizing feature;

FIG. 9 shows a variation of the present invention employing an option for replacement of the inner restraint of FIGS. 7A-7D; and

FIG. 10 shows another variation of the subject delivery system employing another sequential stent release approach.

Variation of the invention from the embodiments pictured is, of course, contemplated.

### DETAILED DESCRIPTION OF THE INVENTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein.

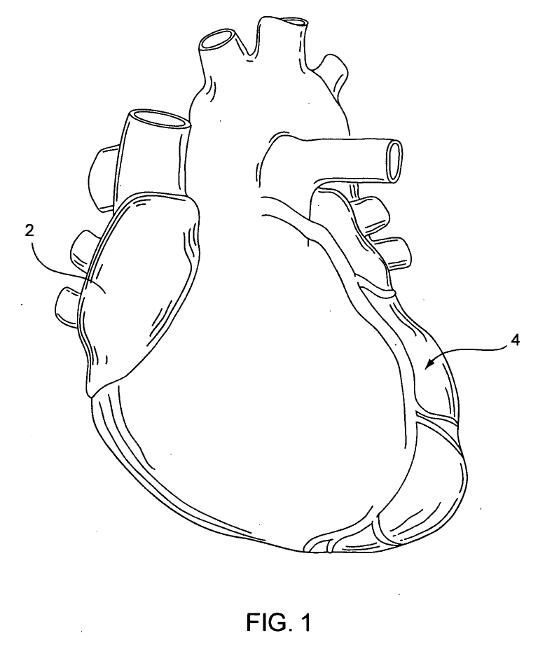

In light of this framework, FIG. 1 shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% coronary percutaneous procedures could be performed with such a system. Such potential offers opportunity for huge gains in human healthcare and a concomitant market opportunity in the realm of roughly $1 billion U.S. dollars—with the further benefit of avoiding loss of income and productivity of those treated.

Features of the present invention are uniquely suited for a system able to reach small vessels (though use of the subject systems s not limited to such a setting.) By “small” coronary vessels, it is meant vessels having a inside diameter between about 1.5 or 2 and about 3 mm in diameter. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations, chronic total occlusions (CTOs), and prevention procedures (such as in stenting of vulnerable plaque).

Assuming a means of delivering one or more appropriately-sized stents, it may be preferred to use a drug eluting stent (DES) in such an application to aid in preventing restenosis. A review of suitable drug coatings and available vendors is presented in “DES Overview: Agents, release mechanism, and stent platform” a presentation by Campbell Rogers, MD incorporated by reference in its entirety. However, bare-metal stents may be employed in the present invention.

While some might argue that the particular role and optimal usage of self expanding stents has yet to be defined, they offer an inherent advantage over balloon expandable stents. The latter type of devices produce “skid mark” trauma (at least when delivered uncovered upon a balloon) and are associated with a higher risk of end dissection or barotraumas caused at least in part by high balloon pressures and related forces when deforming a balloon-expandable stent for deployment.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low pressure balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

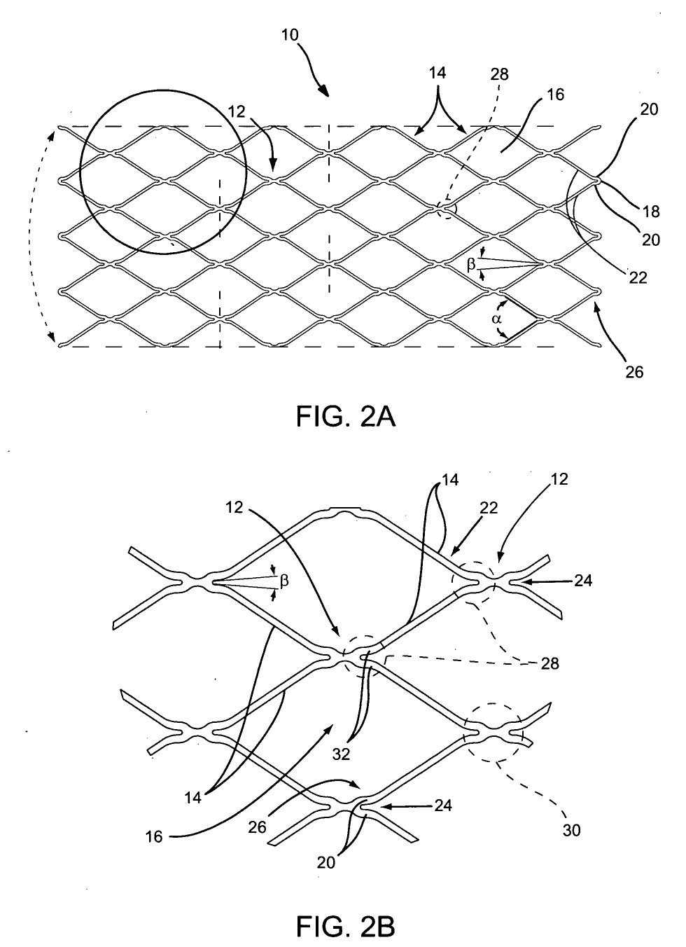

At least some of these noted advantages may be realized using a stent 10 as shown in FIG. 2A. The stent pattern pictured is well suited for use in small vessels. It may be collapsed to an outer diameter of about 0.018 inch (0.46 mm), or even smaller to about 0.014 inch (0.36 mm)—including the restraint/joint used to hold it down—and expanded to a size (fully unrestrained) between about 1.5 mm (0.059 inch) or 2 mm (0.079 inch) or 3 mm (0.12 inch) and about 3.5 mm (0.14 inch).

In use, the stent will be sized so that it is not fully expanded when fully deployed against the wall of a vessel in order to provide a measure of radial force thereto (i.e., the stent will be “oversized” as discussed above). The force will secure the stent and offer potential benefits in reducing intimal hyperplasia and vessel collapse or even pinning dissected tissue in apposition.

Stent 10 preferably comprises NiTi that is superelastic at or below room temperature and above (i.e., as in having an Af as low as 15 degrees C. or even 0 degrees C). Also, the stent is preferably electropolished to improve biocompatibility and corrosion and fatigue resistance. The stent may be a DES unit. The drug can be directly applied to the stent surface(s), or introduced into pockets or an appropriate matrix set over at least an outer portion of the stent. The stent may be coated with gold and/or platinum to provide improved radiopacity for viewing under medical imaging.

For a stent able to collapse to an outer diameter of about 0.012 inches and expand to about 3.5 mm, the thickness of the NiTi is about 0.0025 inch (0.64 mm). Such a stent is designed for use in a 3 mm vessel or other body conduit, thereby providing the desired radial force in the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

In one manner of production, the stent in FIG. 2A is laser or EDM cut from round NiTi tubing, with the flattened-out pattern shown wrapping around the tube as indicated by dashed lines. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost as well as the above-reference effects.

Regarding the finer details of the subject stent, as readily observed in the detail view provided in FIG. 2B, necked down bridge sections 12 are provided between axially/horizontally adjacent struts or arms/legs 14, wherein the struts define a lattice of closed cells 16. Terminal ends 18 of the cells are preferably rounded-off so as to be atraumatic.

To increase stent conformability to tortuous anatomy, the bridge sections can be strategically separated or opened as indicated by the broken lines in FIG. 2A. To facilitate such tuning of the stent, the bridge sections are sufficiently long so that fully rounded ends 18 may be formed internally to the lattice just as shown on the outside of the stent if the connection(s) is/are severed to separate adjacent cells 16. Whether provided as ends 18 or adjoined by a bridge section 12, junction sections 28 connect circumferentially or vertically adjacent struts (as illustrated). Where no bridge sections are provided, the junction sections can be unified between horizontally adjacent stent struts as indicated in region 30.

The advantage of the optional double-concave profile of each strut bridge 12 is that it reduces material width (relative to what would otherwise be presented by a parallel side profile) to improve flexibility and thus trackability and conformability of the stent within the subject anatomy while still maintaining the option for separating/breaking the cells apart.

Further optional features of stent 10 are employed in the cell end regions 18 of the design. Specifically, strut ends 20 increase in width relative to medial strut portions 22. Such a configuration distributes bending (during collapse of the stent) preferentially toward the mid region of the struts. For a given stent diameter and deflection, longer struts allow for lower stresses within the stent (and, hence, a possibility of higher compression ratios). Shorter struts allow for greater radial force (and concomitant resistance to a radially applied load) upon deployment.

In order to increase stent compliance so that it collapses as much as possible, accommodation is made for the stiffer strut ends 20 provided in the design shown in FIG. 2A. Namely, the gap 24 between the strut ends 22 is set at a smaller angle as if the stent were already partially collapsed in that area. Thus, the smaller amount of angular deflection that occurs at ends 20 can bring the sections parallel (or nearly so) when the strut medial portions 22 are so-arranged. In the variation of the invention in FIG. 2A, radiused or curved sections 26 provide a transition from a medial strut angle a (ranging from about 85 degrees to about 60 degrees) to an end strut angle β (ranging from about 30 to about 0 degrees) at the strut junctions 28 and/or extensions therefrom.

In addition, it is noted that gap 24 an angle β may actually be configured to completely close prior to fully collapsing angle α. The stent shown is not so-configured. Still, the value of doing so would be to limit the strains (and hence, stresses) at the strut ends 22 and cell end regions 18 by providing a physical stop to prevent further strain.

In the detail view of FIG. 2B, angle β is set at 0 degrees. The gap 24 defined thereby by virtue of the noticeably thicker end sections 20 at the junction result in very little flexure along those lever arms. The strut medial portions are especially intended to accommodate bending. In addition, a hinging effect at the corner or turn 32 of junction section 28 may allow the strut to swing around angle a to provide the primary mode for compression of the stent.

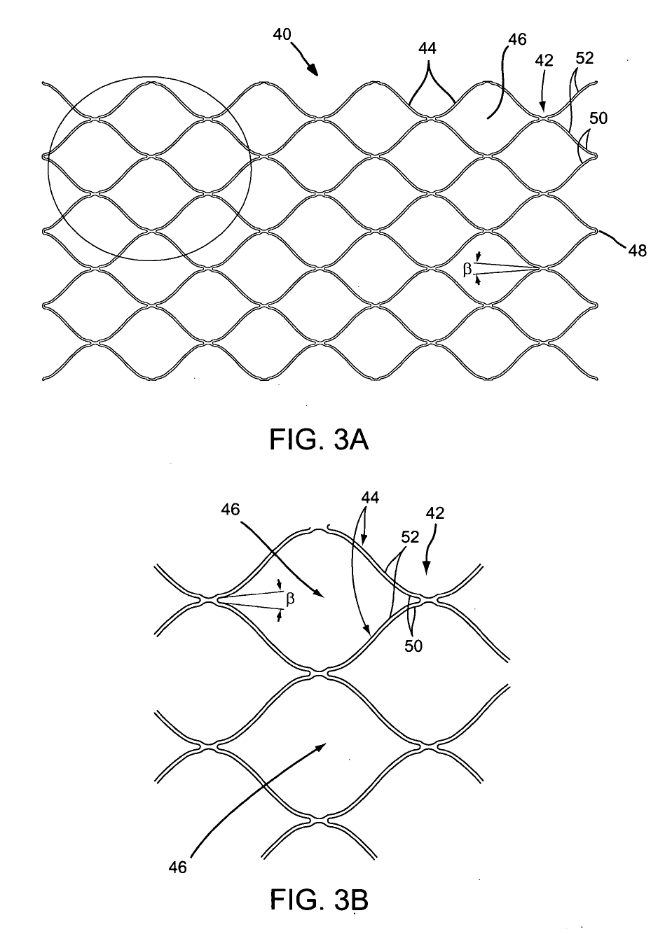

The stent pattern shown in FIG. 3A and detailed in FIG. 3B offers certain similarities as well as some major differences from the stent pattern presented in FIGS. 2A and 2B. As in the variation above, stent 40 includes necked down bridge sections 42 provided between adjacent struts or arms/legs 44, wherein the struts define a lattice of closed cells 46. In addition, terminal ends 48 of the cells are preferably rounded-off so as to be atraumatic.

Furthermore, the bridge sections 42 of stent 40 can be separated for compliance purposes. In addition, they may be otherwise modified (e.g., as described above) or even eliminated. Also, in each design, the overall dimensions of the cells and indeed the number of cells provided to define axial length and/or diameter may be varied (as indicated by the vertical and horizontal section lines in FIG. 3A).

Like the previous stent design, strut ends 50 may offer some increase in width relative to medial strut portions 52. However, as shown in FIG. 3B, as compared to FIG. 2B, the angle β is relatively larger. Such a configuration is not concerned with developing a hinge section and a relatively stiffer outer strut section. Instead, angle β in the FIGS. 3A/3B design is meant to collapse and the strut ends are meant to bend in concert with the medial strut portions so as to essentially straighten-out upon collapsing the stent, generally forming tear-drop spaces between adjacent struts. This approach offers a stress-reducing radius of curvature where struts join, and maximum stent compression.

The “S” curves defined by the struts are produced in a stent cut to a final or near final size (as shown in FIGS. 3A and 3B). The curves are preferably determined by virtue of their origination in a physical or computer model that is expanded from a desired compressed shape to the final expanded shape. So derived, the stent can be compressed or collapsed under force to provide an outer surface profile that is as solid or smooth and/or cylindrical as possible or feasible.

Such action is enabled by distribution of the stresses associated with compression to generate stains to produce the intended compressed and expanded shapes. This effect is accomplished in a design unaffected by one or more expansion and heat setting cycles that otherwise deteriorate the quality of the superelastic NiTi stent material. Further details regarding the “S” stent design and alternative stent constructions as may be used in the present invention are disclosed in U.S. Provisional Patent Application Ser. No. 60/619,437, entitled, “Small Vessel Stent Designs”, filed Oct. 14, 2004 and incorporated herein by reference in its entirety. In the case of each of the above stent designs, by utilizing a stent design that minimizes problematic strain (and in the latter case actually uses the same to provide an improved compressed profile), very high compression ratios of the stent may be achieved from about 5× to about 10× or above.

Delivery systems according to the present invention are advantageously sized to correspond to existing guidewire sizes. For example, the system may have about an 0.014 (0.36 mm), 0.018 (0.46 mm), 0.022 (0.56 mm), 0.025 (0.64 mm) inch crossing profile. Of course, intermediate sizes may be employed as well, especially for full-custom systems. Still further, it is contemplated that the system sizing may be set to correspond to French (FR) sizing. In that case, system sizes contemplated range at least from about 1 to about 2 FR, whereas the smallest known balloon-expandable stent delivery systems are in the size range of about 3 to about 4 FR. In instances where the overall device crossing profile matches a known guidewire size, they may be used with off-the-shelf components such as balloon and microcatheters.

At least when produced in the smallest sizes (whether in an even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in U.S. patent application Ser. No. 10/746,455 “Balloon Catheter Lumen Based Stent Delivery Systems” filed on Dec. 24, 2003 and its PCT counterpart US2004/008909 filed on Mar. 23, 2004, each incorporated by reference in its entirety.

In larger sizes, (i.e., up to about 0.035 inch crossing profile or more), the system is most applicable to peripheral vessel applications as elaborated upon below. Yet, even in “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 and/or 0.025 inch diameter guidewires.

While such a system may not be suitable for reaching the very smallest vessels, this variation of the invention is quite advantageous in comparison to known systems in reaching the larger of the small vessels (i.e., those having a diameter of about 2.5 mm or larger). By way of comparison, among the smallest known over-the-guidewire delivery systems are the Micro-Driver™ by Medtronic and Pixel™ systems by Guidant. These are adapted to treat vessels between 2 and 2.75 mm, the latter system having a crossing profile of 0.036 inches (0.91 mm). A system described in U.S. Patent Publication No. 2002/0147491 for treating small vessels is supposedly capable of downsizing to 0.026 inch (0.66 mm) in diameter. Furthermore, because the core member of the subject device can be used as a guidewire (in one fashion or another) after stent delivery, the present invention offers further advantages in use as elaborated upon below.

As referenced above, it may be desired to design a variation of the subject system for use in deploying stents in larger, peripheral vessels, biliary ducts or other hollow body organs. Such applications involve a stent being emplaced in a region having a diameter from about 3.5 to 13 mm (0.5 inch). In which case, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent patterns shown in FIGS. 2A/2B or 3A/3B.

Again, as a matter of comparison, the smallest delivery systems known to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein.

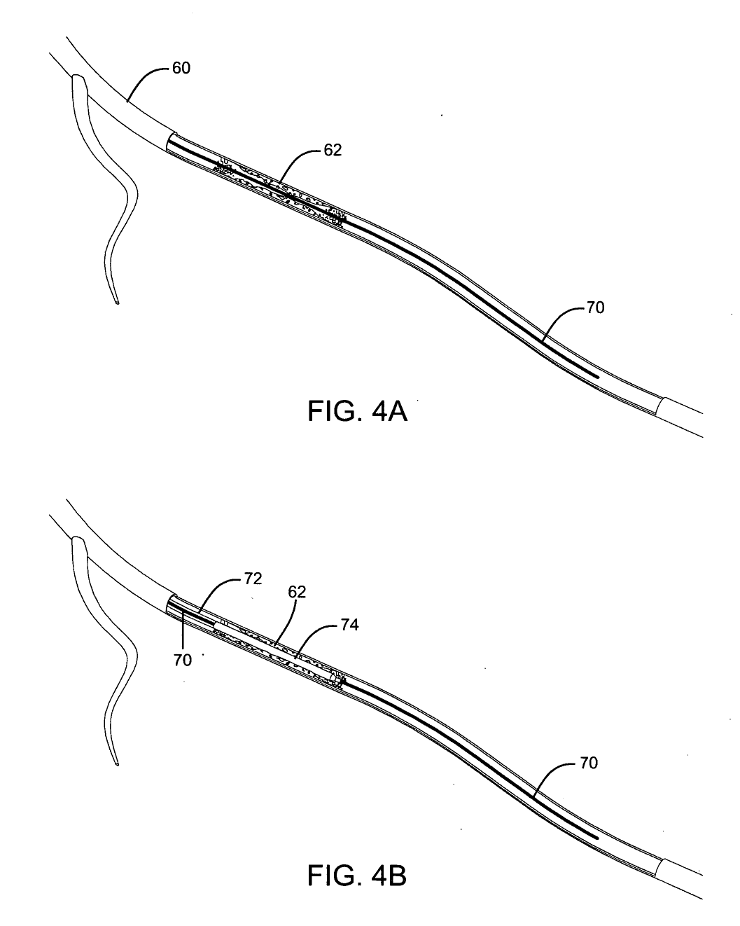

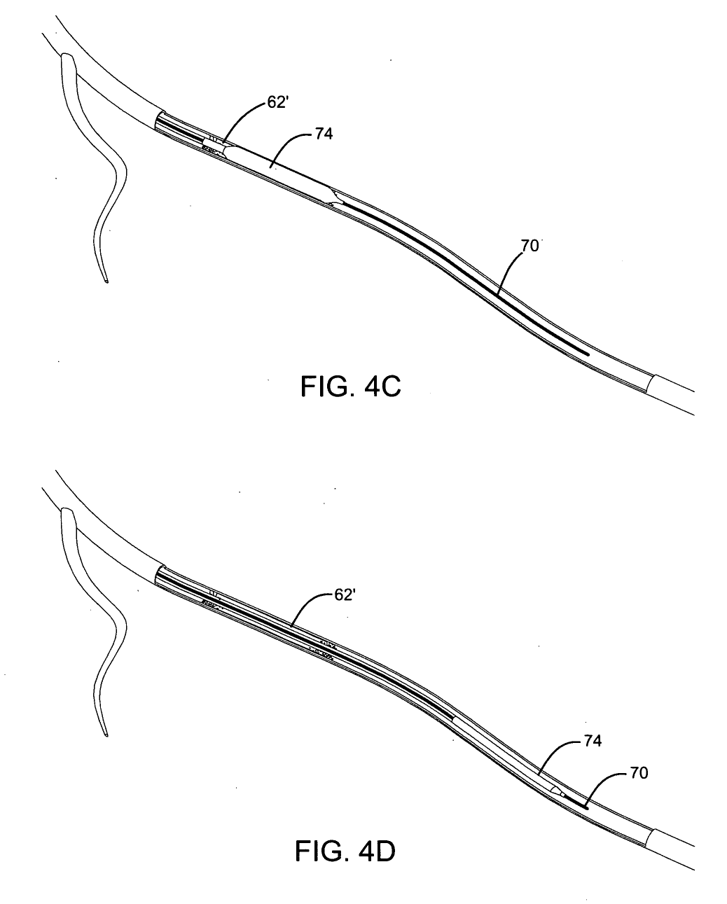

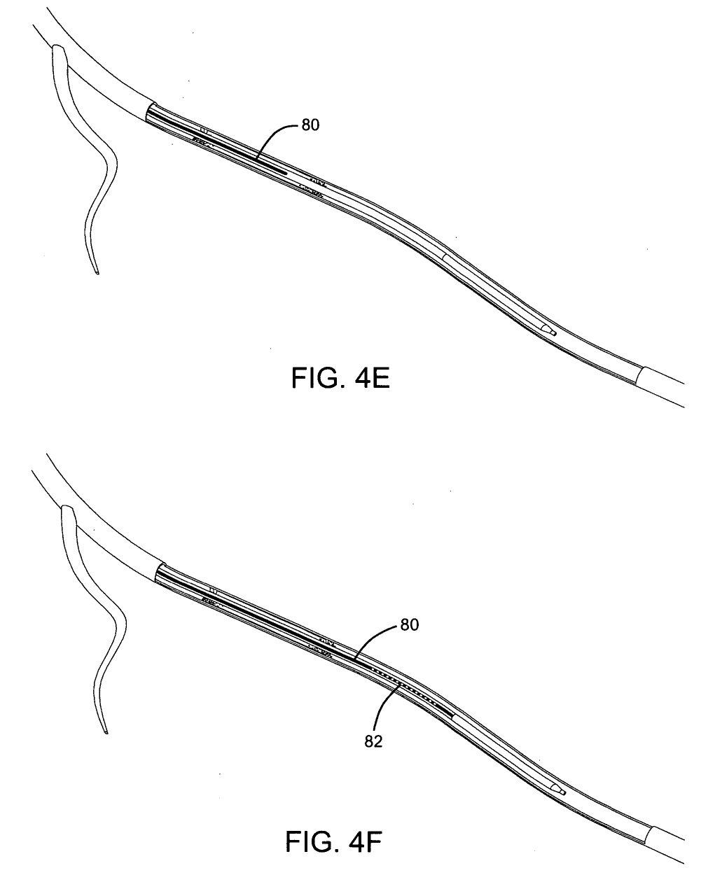

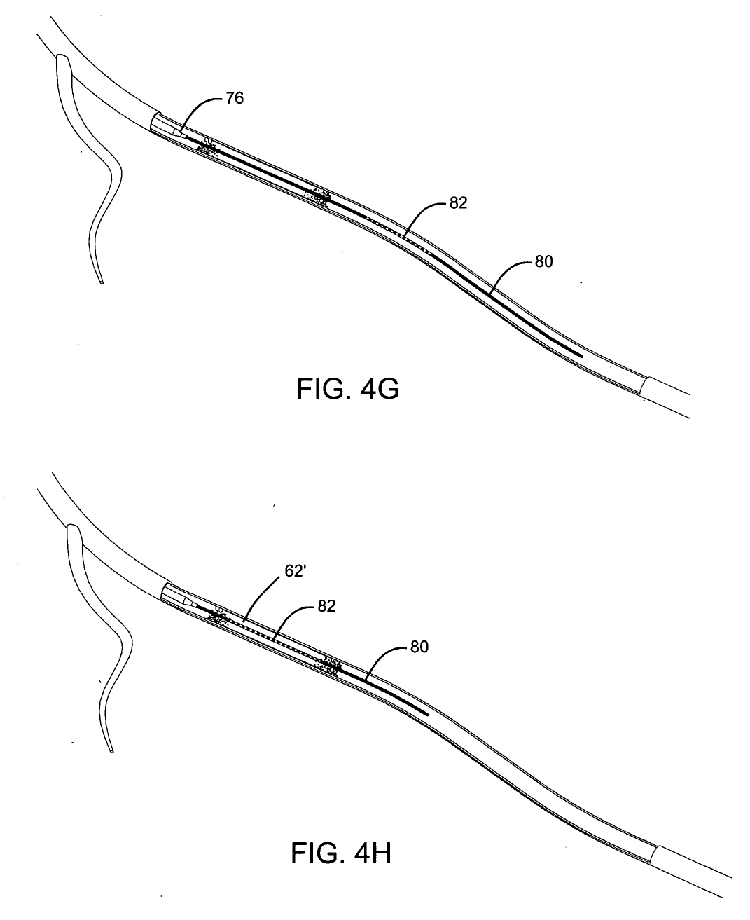

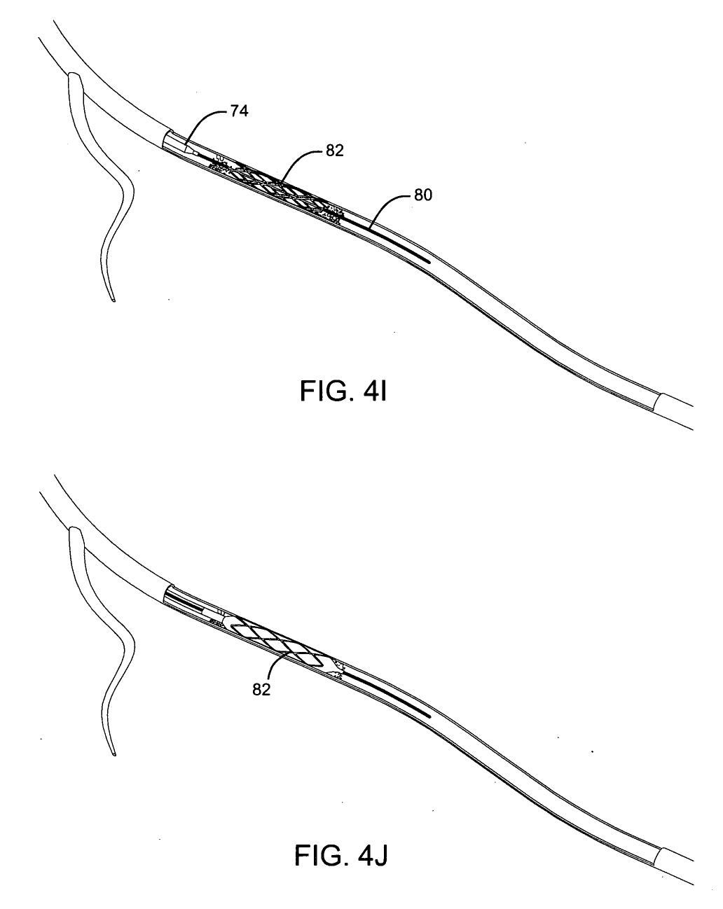

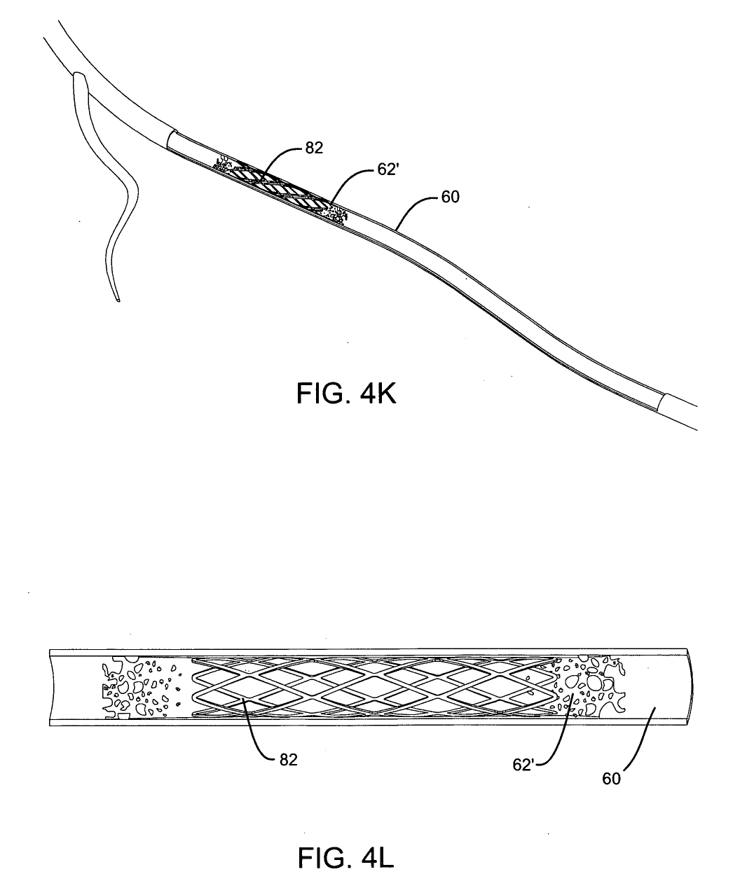

As for the manner of using the inventive system as optionally configured, FIGS. 4A-4L illustrate an exemplary angioplasty procedure. Still, the delivery systems and stents or implants described herein may be used otherwise—especially as specifically referenced herein.

Turning to FIG. 4A, it shows a coronary artery 60 that is partially or totally occluded by plaque at a treatment site/lesion 62. Into this vessel, a guidewire 70 is passed distal to the treatment site. In FIG. 4B, a balloon catheter 72 with a balloon tip 74 is passed over the guidewire, aligning the balloon portion with the lesion (the balloon catheter shaft proximal to the balloon is shown in cross section with guidewire 70 therein).

As illustrated in FIG. 4C, balloon 74 is expanded (dilatated or dialated) in performing an angioplasty procedure, opening the vessel in the region of lesion 62. The balloon expansion may be regarded as “predilatation” in the sense that it will be followed by stent placement (and optionally) a “postdilatation” balloon expansion procedure.

Next, for compatible systems (i.e., systems able to pass through a balloon catheter lumen) the balloon is at least partially deflated and passed forward, beyond the dilate segment 62′ as shown in FIG. 4D. At this point, guidewire 70 is removed as illustrated in FIG. 4E. It is exchanged for a delivery guide member 80 carrying stent 82 as further described below. This exchange is illustrated in FIGS. 4E and 4F.

However, it should be appreciated that such an exchange need not occur. Rather, the original guidewire device inside the balloon catheter (or any other catheter used) may be that of item 80, instead of the standard guidewire 70 shown in FIG. 4A. Thus, the steps depicted in FIGS. 4E and 4F (hence, the figures also) may be omitted.

Alternatively, the exchange of the guidewire for the delivery system may be made before the dilatation step. Yet another option is to exchange the balloon catheter used for predilatation for a fresh one to effect postdilatation.

In addition, there may be no use in performing the step in FIG. 4D of advancing the balloon catheter past the lesion, since such placement is merely for the purpose of avoiding disturbing the site of the lesion by moving a guidewire past the same. FIG. 4G illustrates the next act in either case. Particularly, the balloon catheter is withdrawn so that its distal end 76 clears the lesion. Preferably, delivery guide 80 is held stationary, in a stable position. After the balloon is pulled back, so is delivery device 80, positioning stent 82 where desired. Note, however, that simultaneous retraction may be undertaken, combining the acts depicted in FIGS. 4G and 4H. Whatever the case, it should also be appreciated that the coordinated movement will typically be achieved by virtue of skilled manipulation by a doctor viewing one or more radiopaque features associated with the stent or delivery system under medical imaging.

Once placement of the stent across from dilated segment 62′ is accomplished, stent deployment commences. The manner of deployment is elaborated upon below. Upon deployment, stent 82 assumes an at least partially expanded shape in apposition to the compressed plaque as shown in FIG. 41. Next, the aforementioned postdilatation may be effected as shown in FIG. 4J by positioning balloon 74 within stent 82 and expanding both. This procedure may further expand the stent, pushing it into adjacent plaque—helping to secure each.

Naturally, the balloon need not be reintroduced for postdilatation, but it may be preferred. Regardless, once the delivery device 80 and balloon catheter 72 are withdrawn as in FIG. 4K, the angioplasty and stenting procedure at the lesion in vessel 60 is complete. FIG. 4L shows a detailed view of the emplaced stent and the desired resultant product in the form of a supported, open vessel.

Furthermore, it is to be recognized that the subject invention may be practiced to perform “direct stenting.” That is, a stent may be delivered alone to maintain a body conduit, without preceding balloon angioplasty. Likewise, once one or more stents are delivered with the subject system (either by a single system, or by using multiple systems) the post-dilatation procedure(s) discussed above are merely optional. In addition, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

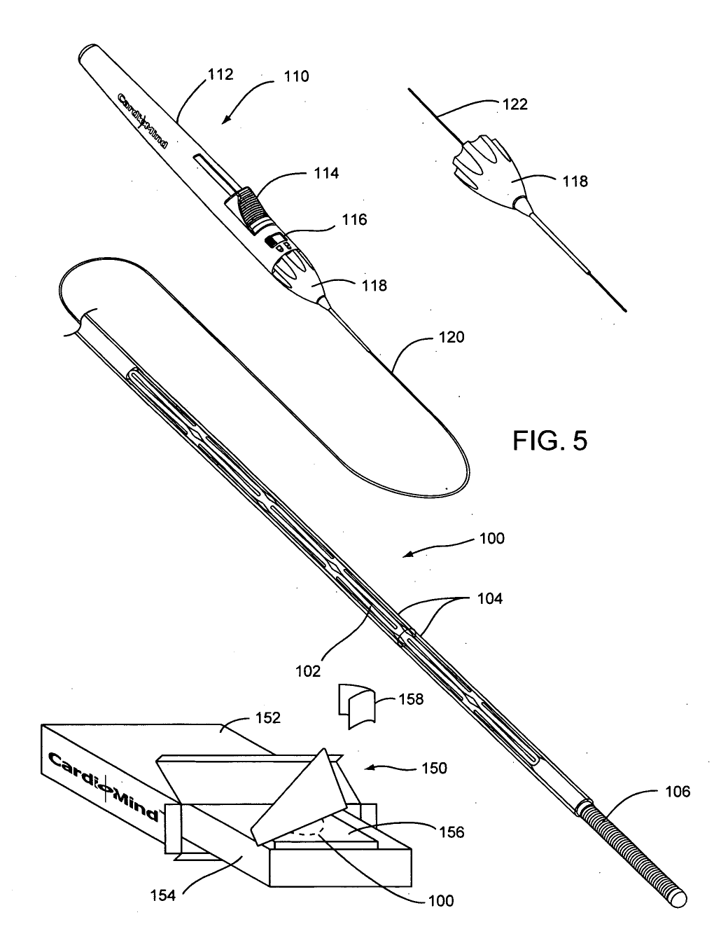

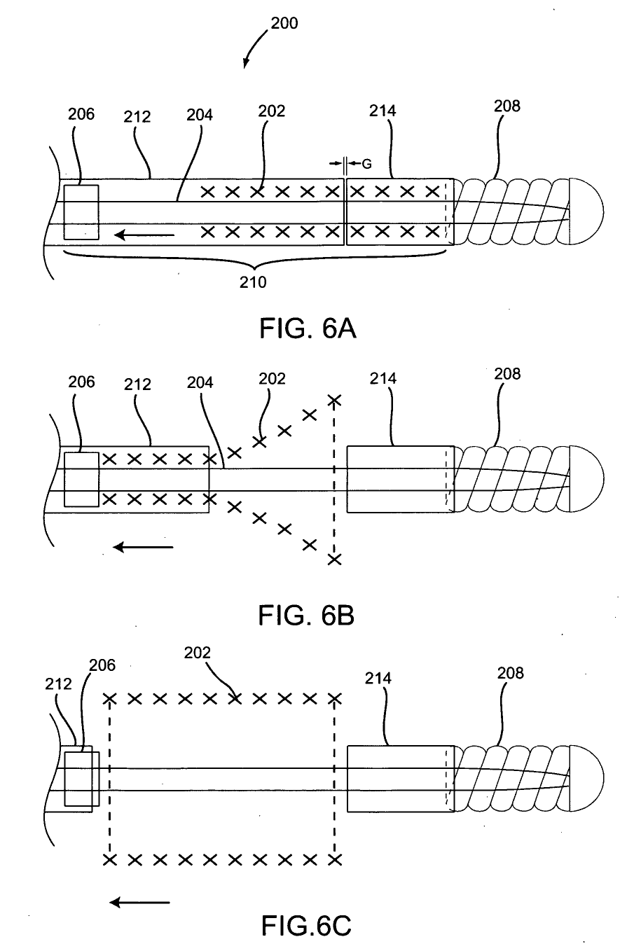

A more detailed overview of the subject delivery systems is provided in FIG. 5. Here, a delivery system 100 is shown along with a stent 102 shown in a collapsed configuration upon the delivery guide member. A tubular restraint assembly 104 is provided over and around the stent to restrain it from expanding. The restraint variation shown in FIG. 5 is elaborated upon in connection with FIGS. 6A-6C; others as discussed further below may be employed in the delivery system as well.

Irrespective of the restraint approach selected, the proximal side of the system may be constructed in the manner of a simple sheath system. In this respect, the inventive system may resemble those described in U.S. Pat. Nos. 6,280,465; 6,833,003, the disclosures of which are herein incorporated by reference, or others. Alternatively, the stent restraint member(s) may be actuated by an internal pull wire or core wire. In such instances, exemplary proximal-side device construction approaches are provided in U.S. Pat. No. 6,736,839 and application Ser. Nos. 10/792,657,10/792,679 and 10/792,684, filed on Mar. 2, 2004, and Ser. No. 10/991,721 filed Nov. 18, 2004, the disclosures of which are herein incorporated by reference.

In any case, the delivery guide preferably comprises a flexible atraumatic distal tip 106 of one variety or another. On the other end of the delivery device, a custom handle 110 may be provided. The body 112 of the device may carry a wheel 114 or other means (such as a trigger, lever arm or slider) for actuating sheath/restraint or core member withdrawal. The delivery device handle may include a lock 116 to prevent inadvertent actuation. Similarly, handle 110 may include various safety or stop features and/or ratchet or clutch mechanisms to ensure one-way actuation.

Furthermore, a removable interface member 118 may be provided to facilitate taking the handle off of the delivery system proximal end 120. The interface may be lockable with respect to the body and preferably includes internal features for disengaging the handle from the delivery guide. Once accomplished, it will be possible to attach or “dock” a secondary length of wire 122 on the delivery system proximal end, allowing the combination to serve as an “exchange length” guidewire, thereby facilitating changing-out the balloon catheter or performing another procedure. Alternatively, a core member within the system may be an exchange-length wire.

FIG. 5 also shows packaging 150 containing at least one coiled-up delivery system 100. When a plurality of such systems are provided (in one package or by way of a number of packages held in stock), they are typically configured in support of a methodology where an appropriate one is picked to reach a target site and deploy a stent without unintended axial movement of the same as per the methodology of Ser. No. 10/792,684, referenced above. Thus, the packaging may serve the purpose of providing a kit or panel of differently configured delivery devices. In the alternative, the packaging may be configured as a tray kit for a single one of the delivery systems.

Either way, packaging may include one or more of an outer box 152 and one or more inner trays 154, 156 with peel-away coverings as is customary in medical device product packaging. Naturally, instructions for use 158 may also be provided. Such instructions may be printed product included within packaging 150 or be provided in connection with another readable (including computer-readable) medium. The instructions may include provision for basic operation of the subject devices and associated methodology.

In support of such use, it is to be understood that various radiopaque markers or features may be employed in the system to 1) locate stent position and length, 2) indicate device actuation and stent delivery and/or 3) locate the distal end of the delivery guide. As such, various platinum (or other radiopaque material) bands or other markers (such as tantalum plugs) may be incorporated into the system. Especially where the stent employed may shorten somewhat upon deployment, it may also be desired to align radiopaque features with the expected location (relative to the body of the guide member) of the stent upon deployment. For such purposes, radiopaque features may be set upon the core member of the delivery device proximal and distal of the stent.

Turning now to FIGS. 6A, 6B and 6C, these show an exemplary embodiment of a single user input action stent delivery system of the subject invention. The figures show a stent delivery system 200 that includes a stent 202 held in a collapsed configuration upon an inner member 204 which may be a wire such as a basic core wire. Alternatively, the stent may be carried on an extension section as described in U.S. patent application Ser. No. 10/991,721, noted above. The stent is typically a self-expanding stent.

A stent stop section or stent blocker 206 adapted to abut the proximal end of the stent is carried by, connected to or integrally formed with the inner member 204. Inner member 204 also includes a flexible atraumatic tip 208.

A tubular restraint assembly 210 is,provided over and around the stent to hold the stent in a reduced-diameter configuration and restrain it from expanding. Restraint assembly 210 is a two-piece construction and includes two tubular restraining members: a first, proximal tubular restraining member 212 and a second, distal tubular restraining member 214.

The proximal restraining member is slideable or moveable over inner member 204 (and stent 202) and the distal restraining member is not slideable (i.e., is stationary, fixed or connected a distal end to a section of the delivery device body). The distal restraining member may be attached to the inner member, the coil tip, etc. in any suitable manner.

To deploy stent 202, it is freed from the restraining assembly members so that is may expand from a reduced-diameter configuration to an increased-diameter configuration. Specifically, to deploy stent 202 proximal restraining member 212 is slideably withdrawn backwards over the stent, or rather is moved in a proximal direction (in the direction of the arrow of FIG. 6A), causing the stent to slide proximally so that the portion of the stent that is covered by the distal restraining member is released therefrom and the proximal end of the stent is caused to abut stent stop or stent blocker 206, as shown in FIG. 6B.

As the proximal restraining member is slid proximally, the distal restraining member is maintained in a fixed position at the distal end of the delivery device body. As shown in FIG. 6B, once unrestrained by the distal restraining member, the portion of the stent released from the distal restraining member is free to expand from a reduced-diameter configuration to an increased-diameter configuration. Then, with the proximal end of stent 202 abutting blocker or stop 206, the proximal restraining member is drawn off of the stent so that the stent is completely free of the restraint as shown in FIG. 6C.

Accordingly, the single user input action of sliding the proximal restraining member in a proximal direction effects a two-stage stent deployment protocol: 1) releasing a first or distally-restrained portion of the stent from a distal restraining member using the proximal restraining member without having to overcome stentrestraint friction over the proximal end of the stent, and 2) releasing the remainder or proximally-restrained portion of the stent from the proximal restraining member. In this manner, stent release is accomplished in a distal-to-proximal fashion. Because the restraining system includes two members—neither of which covers the full length of the stent, and only one of which is required to slide over the stent at a time, stent delivery forces are broken-up and decreased actuation force is required to deploy the stent as compared to a restraining member that covers the full length of the stent.

This two-piece restraining assembly is configured so that first restraint 212 restrains a first portion of stent 202 and second restraint 214 restrains a second portion of stent 202. Typically, prior to withdrawal of the proximal restraining member for stent deployment, the restraining members will (together) generally extend over about 100% of the length of the stent. For example, restraining members 212 and 214 may abut each other in a pre-deployment configuration (i.e., cover the entire length of the stent). In certain variations, some overlap or, conversely, a gap “G” between the members may exist (e.g., up to about 10% to about 25% the length of the stent or more), as shown in FIG. 6A. Indeed, a gap section may offer further benefits in terms of force-reduction by reducing stent coverage by one or each of the restraining members.

The invention variation shown in FIGS. 6A-6C includes a restraining member assembly in which one of the members holds down a greater length of the stent than the other restraining member. Typically, the proximal restraining member or the restraining member that is slideable over the stent during stent deployment extends over a majority of the stent's overall length and the distal restraining member or the restraining member that is fixed in place extends over the remaining length of the stent. Without involving other parameters, the greater coverage by the proximal restraint results in maintaining a static friction state upon withdrawing that member and sufficient break-away force to cause the stent portion in the distal retraint member to be allowed to slide under a dynamic friction load out of that member.

For example, a first restraining member (e.g., the proximal restraining member) may cover about 60% (or more) of the length of the length of the stent and a second restraining member that covers about 40% (or less) of the length of the stent, prior to stent deployment (i.e., prior to withdrawing the first restraining member). This “60/40” stent delivery system is illustrated in FIGS. 6A-6C in which, prior to deployment of the stent, proximal restraining member 212 extends over about 60% of the length of stent 202 and distal restraining member 214 extends over about 40% of the length of stent 202. Other variations are possible as well. For example, continuing with the convention of “the percentage of the stent's length covered by the proximal restraining member/ the percentage of the stent's length covered by the distal restraining member”, variations may include, but are not limited to, “70/30”, “80/20”, and “90/10” stent delivery systems.

With these larger spreads in coverage, greater predictability or insurance of intended actuation is realized. However, the force break-up advantages decrease. A more advantageous system from a force perspective may be a 54/45 system. In certain instances, such a differential of 10% the length of he stent coverage or even 5% (e.g., in a 52.5/47.5 system) may be adequate to ensure predictable withdrawal of the stent from the distal restraint member, followed by ultimate deployment from the proximal restraint portion.

Moreover, by employing a gap “G” as described above, even more advantageous 50/45 (with a 5% gap), 45/40 (with a 10% gap) or 40/30 (with a 20% gap) systems—from the perspective of reducing actuation forces (for each of the first and second stage actuations) may be employed. Other exemplary systems may, of course, be constructed according to the principles illustrated above.

Further variability contemplated in the invention includes variation of the inner diameters of the restraining members. These diameters may be the same or may differ. For example, in certain embodiments the inner diameter of the proximal restraining member may be less than the inner diameter of the distal restraining member. This may facilitate holding the proximal end of the stent with the proximal restraint portion tighter and more predictably releasing the distal end of the stent from the distal restraining member upon proximal restraint portion withdrawal. In fact, with systems in which the inner diameter of the proximal restraining member is less than the inner diameter of the distal restraining member, the proximal restraining member may even cover relatively less of the stent while still offering predictable actuation. For example, such a system may be offer a 50/50 ratio of proximal to distal restraint section length. Alternatively, less than about 50% of the length of the restraining system may be covered by the proximal restraining member.

Any of the restraining members of any of the systems described herein may include a lubricious coating to further decrease friction. For example, an inner surface (i.e., a stent-facing surface) and/or outer surface of a restraining member may be coated or covered by a polymeric material (e.g., with a lubricious material such as TEFLON®, i.e., PolyTetraFluoroEthelyne—PTFE) or otherwise. The restraint sections may comprise hypotubing, high-strength polymeric tubing (e.g., Polyamide) or a reinforced or composite structure such as described in U.S. Patent Application No. 60/690,937 filed Jun. 14, 2005 and incorporated by reference in its entirety.

The restraint member portions may each have the same level of lubricity, or they may differ. Specifically, it may be desirable that the distal restraint section is more lubricious to facilitate the stent sliding out of that body. The implications for such a system are like those discussed above in which restraint section diameter (particularly inner diameter) are modified to assist in tuning the system for desired actuation. Therefore, any one of surface treatment, length and/or diameter of the restraint portions may be employed in tuning the system to effect the subject methodology.

In another aspect of the invention, a multi-piece restraint may include a radially expandable restraint member. Though accomplished in a different manner than from those variations of the invention just described, these additional variations are also configured to moderate frictional forces between the restraining member(s) and the stent. Likewise, both types of systems rely on a multi-piece restraint assembly in which withdrawal of the proximal restraint member slides the stent (typically self-expanding) until it abuts a stop feature wherein continued proximal movement of the restraint frees the remainder of the stent.

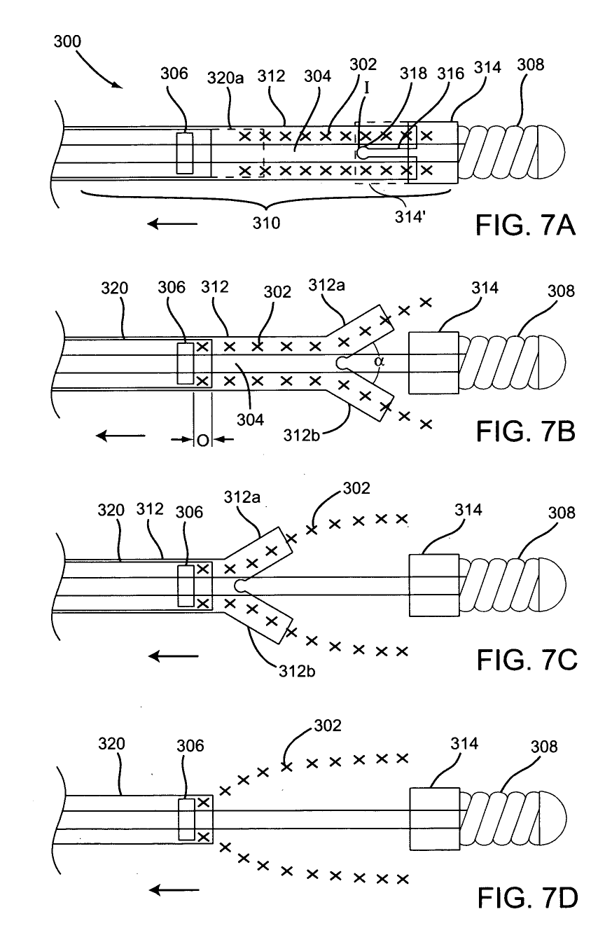

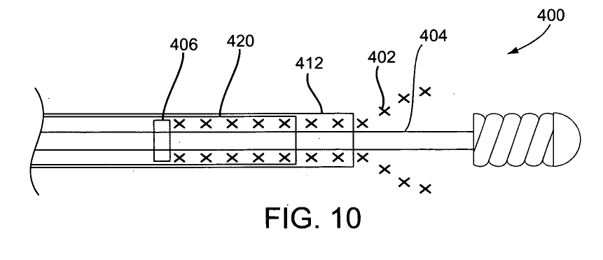

The variations of the invention shown in FIGS. 7A-7D however, employ proximal movement of the restraint to unlatch or free a radially expandable proximal restraint member from a distal restraint member. Once freed, the stent is allowed to expand from its reduced-diameter configuration to its increased-diameter configuration. Expansion of the stent alone may open the proximal restraint, or the restraint may be self-expanding.

More specifically, FIG. 7A shows a stent delivery system 300 that includes a stent 302 held in a collapsed configuration upon an inner member 304, such as a wire (e.g., a basic corewire), which inner member includes stent stop section or stent blocker 306 adapted to abut the proximal end of the stent, and flexible atraumatic tip 308.

Restraint assembly 310 includes a first or proximal self-expanding, radially expanding or expandable restraining member 312 and a second or distal tubular restraining member 314. The distal restraining member is adapted to receive and hold down a distal portion of proximal restraint 312 to prevent radial expansion thereof until the slideable withdrawal of expandable restraining member 312 (together with the stent) from the distal restraining member for stent deployment.

The lengths of the proximal and distal restraining members may vary. Often, the proximal restraining member holds down at least a majority of the length stent 302. It may constrain less, as per the above, in instances where the lesser overlap is still adequate to grip the stent and slide it proximally with the near-side or proximal restraint 312. The distal restraining member may be only so long as required to secure the proximal restraint section in a closed position. In other words, it may be as short as about 0.005 to about 0.02 inches long for a 0.014 crossing profile system. Alternatively, the distal restraining member may be longer as indicated by dashed-line 314′ of FIG. 7A. When longer, greater security may be offered by an enlarged overlap region—notwithstanding with the potential result in increased actuation/withdrawal forces as the outwardly-straining proximal restraint section must slide past (after break-away from or overcome a static friction condition) a greater surface area of material. Alternatively, the proximal restraining member may be shortened. This measure may be desirable from the perspective of reducing the length of exposed material sliding past tissue in the most tortuous, most distal anatomy.

In any event to deliver stent 302, radially expandable restraining member 312 is slideable or moveable relative to inner member 304 (first with the stent) in a proximal direction indicated by the arrows shown in the figures, and distal restraining member 312 is stationary. The distal restraining member may be attached to the inner member, the coil tip, etc. in any suitable manner.

Once released from the distal restraining member 314, radially-expandable restraining member 312 opens to its increased-diameter configuration, as shown in FIGS. 7B and 7C. The distal end of the restraining member may spring open by its own action, or be pushed or deformed open by the action of the stent. Withdrawal of the radially expandable restraining member 312 causes the proximal end of stent 302 to abut blocker 306. In a variation of the invention shown, an optional partial restraining member 320 adapted to capture the proximal end of the stent is offered to prevent the stent from bypassing the blocker, as described in greater detail below.

As shown in FIG. 7D, the final stages of stent deployment include completely freeing the stent from the radially expandable restraining member. In certain variations, the partial restraint 320 may be withdrawn to effect such action. Alternatively, corewire 304 with blocker 306 may be nudged forward a small amount (without risking vessel perforation) to free the stent from the partial restraint.

The degree to which radially expandable restraining member 312 opens may vary. In certain variations of the subject invention, radially expandable restraining member 312 opens only at a distal end portion of the restraint (as shown in the figures), thereby aiding in initially pulling-off the restraint. Alternatively, the expandable restraint may open over a greater length—even over the full length of the stent. In at least the latter case, two or more restraint segments will be produced that are to be drawn out from between a vessel wall and the stent complete the subject medical procedure.

Radially expandable restraining member 312 typically includes slits or grooves 316 cut into the body of the radially expandable restraining member, the number of which will depend on the desired number of expandable restraint segments. In the variations shown in the figures, two slits provide two expandable segments or legs 312a and 312b. When the proximal restraint section comprises a stronger or stiffer material, it may be appropriate to provide upwards of about 3, 4, 5 or 6 segments that open up and separate (at least partially) upon withdrawal from distal restraint section 314. Where “grooves” or scored sections are provided, they may break-up or apart upon release from distal, latching restraint 314.

The lengths of the scores, slits, etc. of proximal restraint section 312 may vary and depend on the particular configuration of the system and/or restraint material properties, where slits of a given radially expandable restraining member may have the same or different lengths. In certain variations, the slits may advantageously have a length that ranges from about 10% to about 100% of the length of the stent. More typically, the slits may have a length from about 25% to 50% of the length of the stent. Shorter lengths offer the advantages of this aspect of the invention to a lesser degree, whereas longer lengths may not retain their native shape or geometry to best restrain radial expansion of a stent.

To reduce or minimize the localized concentration of stress at the intersection “I” of expandable restraint segments 312a and 312b, the restraint slits may include a stress relieving feature 318, e.g., in the form of a stress relieving notch or circular or semi-circular hole or the like. In order that the restraint have sufficient strength to confine the stent and remain locked in place, it may comprise a high-strength polymer such as PEEK or Polyimide. Alternatively, a superelastic NiTi construction may be employed, particularly one in which the interior of the metal is coated or lined with PTFE. Still further, construction techniques as described in U.S. patent application Ser. No. 11/147,999 filed Jun. 7, 2005 and entitled, “Ten-thousands Scale Metal Reinforced Stent Delivery Guide Sheath or Restraint,” which application is incorporated by reference may be employed. Naturally, any of the other restraint member sections may be so-constructed.

The degree to which the restraint segments open may vary. They may open to angle α (as shown in FIG. 7B) ranging from about 10 to about 120 degrees, or otherwise. The intent is that in their opening that the stent is able to at least partially expand in the relevant region.

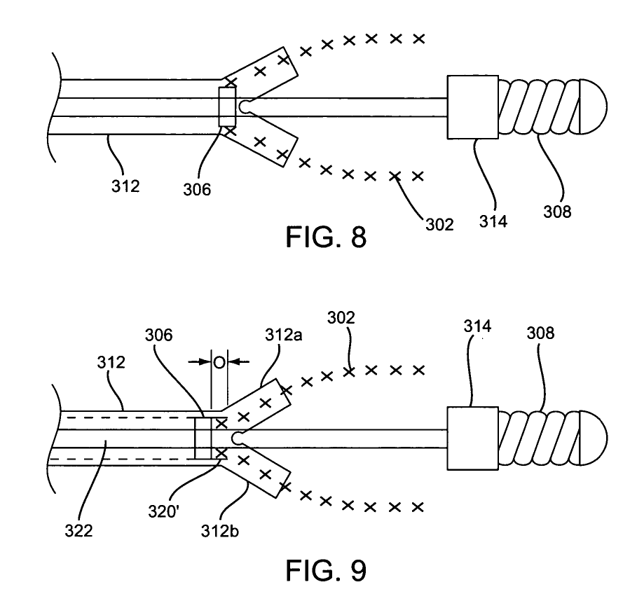

As noted above, an optional partial or inner restraining member (e.g., in the form of a partial-length sheath 320, band or the like) for capturing the proximal end of the stent may be provided. In this manner, the stent is prevented from sliding past the stent blocker 306 during withdrawal of the expanded or open restraining member 312 as illustrated in FIG. 8. Such a result may be less desirable from a perspective of stent positioning and deployment. However, it may be effective in stent delivery by relying on a stentivessel wall interface developed at a distal end of the stent when it opens to allow sufficient grip of the stent to complete restraint member 312 withdrawal.

The partial restraint may be configured in a number of ways. In certain variations, the partial restraint may comprise a band or mini-sheath 320 as shown in FIGS. 7A-7D. Therefore, to effect final stent release the mini-sheath will be pulled back to free the most proximal portion of the stent. Alternatively, in certain variations the partial restraint may comprise an extension of blocker 306, as shown in FIG. 9, such that blocker 306 includes a partial restraining portion 320′ (i.e., the partial restraint is an integral extension of the stent blocker). When of this type, core member 322, together with the blocker 306 and band 320′, is withdrawn to effect final release of the stent with reliance placed on the stent/vessel wall interface to stabilize the stent position. Alternatively, the band 320′ may be configured so that the stent squeezes itself out of its grip when the proximal restraining member 312 is sufficiently withdrawn.

In either variation of the invention shown in FIGS. 7A-7D or FIG. 8, in order to stabilize the stent so that it does not slide over the blocker during withdrawal of the radially expandable restraining member 312, the length or overlap “O” of the partial restraint (whether a partial-length sheath 320 or an extension segment 320′ from a blocker) with the stent in this section may be as little as about 0.050 inches or as much as about 0.25 inches, in other terms, it may be from about 1/20 to about ¼ the length of the stent.

Longer length sections may, however, be desired for other reasons. Namely, to further reduce amount of the stent's length that the expandable restraining member has to hold down, the partial restraint may be elongated as shown in broken-line 320a in FIG. 7A. For example, prior to sliding the expandable restraining member and stent in a proximal direction to release the same from the distal restraint, the partial restraint may overlap the proximal portion of the stent at least about 10% or more (e.g., may overlap about 10% to about 50% of the stent).

This system discussed directly above relates to another system according to the present invention. Like all the systems described above, the delivery system shown in FIG. 10 is configured to moderate frictional forces between the restraining member(s) and the stent by relying on a multi-piece restraint approach to break up-the forces experienced in actuating the individual members.

Stent delivery system 400, shown with the stent partially deployed, includes a stent 402 held in a partially compressed configuration upon an inner member 404, such as a corewire. Inner member 404 includes stent stop section or stent blocker 406 integral therewith or connected thereto and flexible atraumatic tip 408.

The multi-piece restraint of stent delivery system 400 shown in FIG. 10 includes a first or proximal restraining member 412, a second or distal restraining member 414. Yet, one or more other intermediate restraining member (not shown) may be provided as well. Factors to consider when adding multiple ones of said members include their difference in diameter as effect upon overall system diameter. For this reason, the use of only two or three restraint members may be preferred in providing the smallest diameter systems. A greater number of partial restraint members increases system complexity.

As shown, outer restraining member 412 and inner restraining member 420 differ in that restraining member 412 extends over a greater length (generally to the end) of the stent and restraining member 420 extends over a lesser length of the stent (i.e., a shorter distance over the length of the stent), prior to withdrawal of the restraining members. To deploy the stent, restraining member 412 is first proximally withdrawn so that only the portion of the stent it radially restraints is released. Restraint member 420 is then proximally withdrawn to complete stent release and deployment.

When the restraining members are not linked, this may require individual withdrawal steps to effect overall stent release. However, the members may each have a proximal end with interface features (not shown) such that withdrawal of an outer member will proceed until it catches an inner member, at which time further withdrawal of the outer member will also result in inner member withdrawal. The details for construction of such a system are within the ordinary level of skill of those in the art.

Methods

The methods herein may be performed using the subject devices or by other means. The methods may all comprise the act of providing a suitable device. Such provision may be performed by the end user. In other words, the “providing” (e.g., a delivery system) merely requires the end user obtain, access, approach, position, set-up, activate, power-up or otherwise act to provide the requisite device in the subject method. Methods recited herein may be carried out in any order of the recited events which is logically possible, as well as in the recited order of events.

Variations

Exemplary aspects of the invention, together with details regarding material selection and manufacture have been set forth above. As for other details of the present invention, these may be appreciated in connection with the above-referenced patents and publications as well as generally know or appreciated by those with skill in the art.

The same may hold true with respect to method-based aspects of the invention in terms of additional acts as commonly or logically employed. In addition, though the invention has been described in reference to several examples, optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Except as specifically defined herein, all technical and scientific terms used herein are to be given as broad a commonly understood meaning as possible while maintaining claim validity.

We claim:

1. A stent delivery system comprising:
a self-expanding stent; and
a delivery guide comprising proximal and distal stent restraining members holding the stent in a reduced-diameter configuration, and a stent abutment feature to stabilize the stent;
wherein withdrawal of the proximal restraining member causes the release of the stent from the distal restraining member and from the proximal restraining member.

2. The stent delivery system of claim 1, wherein the proximal restraining member extends over at least a majority of the length of the stent prior to withdrawal of the proximal restraining member.

3. The stent delivery system of claim 1, wherein the proximal restraining member extends over about 60% or more of the length of the stent in a collapsed configuration.

4. The stent delivery system of claim 3, wherein the distal restraining member extends over about 40% or less of the length of the stent in a collapsed configuration.

5. The stent delivery system of claim 3, wherein the proximal restraining member extends up to about 90% of the length of the stent.

6. The stent delivery system of claim 5, wherein the distal restraining member extends over about 40% to about 10% of the length of the stent.

7. The stent delivery system of claim 1, wherein the proximal restraining member is radially expandable and at least a portion of the proximal restraining member is engageable with the distal restraining member to restrain it from radially expanding.

8. The stent delivery system of claim 7, wherein the proximal restraining member comprises a plurality of longitudinal slits that define the portion of the proximal retraining member that radially expands.

9. The stent delivery system of claim 8, wherein the slits extends about 1/10 to about a full length of the stent.

10. The stent delivery system of claim 9, wherein the slits extend about ⅕ to about ½ the length of the stent.

11. The stent delivery system of claim 10 wherein the slits extend about ¼ to about ⅓ the length of the stent.

12. The stent delivery system of claim 7, further comprising an inner restraining member adapted to capture the proximal end of the stent as the proximal restraining member is withdrawn.

13. The stent delivery system of claim 9, wherein the inner restraining member is adapted to overlap with the stent by about 0.05 to about 0.25 inches.

14. The stent delivery system of claim 1, further comprising an inner member over which the stent is collapsed.

15. The stent delivery system of claim 1, wherein the inner member comprises a corewire.

16. The stent delivery system of claim 1, further comprising a stent stop to abut a proximal end of the stent.

17. A stent delivery system comprising
a self-expanding stent; and
a delivery guide comprising an inner member, a radially expandable proximal restraining member holding the stent collapsed over the inner member, and a distal restraining member restraining the expansion of the radially expandable proximal restraining member, and a stent abutment feature, the stent abutment feature separated from the stent a distance great enough to allow the stent to translate over the inner member with the proximal restraining member for release from the distal restraining member before withdrawal of the proximal restraining member from the stent.

18. The stent delivery system of claim 17, wherein the proximal restraining member is adapted to radially expand over substantially a full length of the stent.

19. The stent delivery system of claim 17, wherein the proximal restraining member is adapted to radially expand over substantially less than a full length of the stent.

20. The stent delivery system of claim 17, further comprising an inner restraint adapted to maintain the stent at the stent abutment feature.

21. (canceled)

22. A method of delivering a stent, the method comprising:
advancing a stent delivery system to a target location within a vessel;
removing a first restraint portion from the stent, without removing a second restraint portion from the stent; and
next removing the second restraint portion from the stent.

23. The method of claim 22, wherein the stent delivery system selected from one described in claims 1, 17 and 18.

24. The method of claim 22, wherein the fist and second removing is accomplished by a single user action.

25. The method of claim 24, wherein the stent delivery system is selected from one described in claims 1, 17 and 18.
